(19) United States
(12) Patent Application Publication
(10) Pub. No.: US 2005/0220749 A1
(43) Pub. Date: Oct. 6, 2005

(54) Cosmetic product for the protection of the skin or of phaneres

(75) Inventors: Antoine Gedouin, Saint-Coulomb (FR); Romuald Vallee, Saint-Meloir-des-Ondes (FR); Pierre-Yves Morvan, Rennes (FR)

Correspondence Address: WESTERMAN, HATTORI, DANIELS & ADRIAN, LLP, 1250 CONNECTICUT AVENUE, NW, SUITE 700, WASHINGTON, DC, 20036, US

(73) Assignee: CODIF INTERNATIONAL SAS, Saint-Malo Cedex (FR)

(21) Appl. No.: 10/497,914

(22) PCT Filed: Nov. 28, 2002
(86) PCT No.: PCT/FR02/04100

§ 371 Date: Jun. 7, 2004

(30) Foreign Application Priority Data
Dec. 7, 2001 (FR) 01/16176

(51) Int. Cl. A61K 7/06; A61K 35/78

(52) U.S. Cl. 424/74; 424/765

(57) ABSTRACT

The invention concerns a cosmetic product to protect the skin or phaneres. The cosmetic product according to the invention is characterized in that it contains an extract of at least one of the products obtained during valorization of the apple.

The present invention concerns a cosmetic product for the protection of the skin or of the phaneres. More specifically, such a cosmetic product has an anti-radical activity.

Such a cosmetic product having an anti-radical activity possesses generally a vital power for the cells of the skin as well as a protective effect for said cells.

It has been shown that a cosmetic product that would contain an extract of at least one of the products obtained from valorization of the apple would have an anti-radical effect.

The process of making cider is recalled below, so as to give prominence to what is called, in the present description, products obtained during valorization of the apple.

This process of making cider is as follows. The apples are ground so as to extract from them a juice, which is called must. The excess of apples can be recuperated to extract from it the pectin. The latter is already known for its cholesterol-lowering properties.

The must is transferred to a fermenter where it is subjected to a double fermentation: the first, an alcoholic fermentation, transforms the sugar of the apple juice into ethanol and into higher alcohols, whereas the second is a malolactic fermentation.

The must will then be subjected to a sediment clearing opearation resulting from pectins that trap the nitrogenated materials and the parasitic yeasts contained in the juice. These materials and these yeasts are carried along to the surface by the carbon dioxide generated by the fermentation, so that a brown and slimy foam forms at the surface of the must, which is called “brown cap”. Its formation can be facilitated by the introduction of calcium chloride. Further, at the bottom of the fermenter, sediment accumulates.

Traditionally, the must alone, which has clarified during the previous step, generally called defecation, is then extracted. This must, which has been placed in another fermenter, thus keeps fermenting until a point of equilibrium where the obtained liquid, which is the desired cider, is bottled.

Industrially, fermentation is continuous and the different elements that constitute the must are separated by centrifugation and filtering. Thus, the cider is obtained and bottled.

It is possible to obtain a cider vinegar from this cider by letting it age in contact with a mother. Cider vinegar is known for its cosmetic properties.

All in all, the products which can be obtained during valorization of the apple are as follows:
the marc, residue of apples that have been pressed to extract their juice. Nowadays, the marc is essentially not valorized except to recuperate pectin.
the juice extracted from the apple, called must,
the products of fermentation of the must, which are:
   the must strictly speaking, which, fermented over a relatively large period of time, will finally give cider,
   the sediment,
   the brown cap.

Thus, the present invention concerns a cosmetic product which is characterized in that it contains an extract of at least one of the products obtained during valorization of the apple, such as the marc constituted by residues of apples that have been pressed to extract their juice, one of the products of the fermentation of the must, which can be the must strictly speaking after alcoholic fermentation, the must strictly speaking after malolactic fermentation or the sediment obtained after alcoholic fermentation of the must, or the brown cap obtained after alcoholic fermentation of the must.

This cosmetic product is a cream, a serum, a gel, a shampoo, a bath, a micro-emulsion or a lotion.

The present invention concerns also the use of an extract of at least one of the products obtained during valorization of the apple for the manufacture of a cosmetic product possessing a vital power for the cells of the skin as well as a protective effect for said cells. Said product or one of said products obtained during valorization of the apple is one of the following products: the marc, the must after alcoholic fermentation, the must after malolactic fermentation, the sediment, the brown cap, the cider, the cider vinegar.

An anti-radical activity of a cosmetic product obtained by using a product obtained from valorization of the apple, here unpasteurized brut bottled cider, is shown below.

This anti-radical activity has been demonstrated by a method based on the use of a hypoxanthin-xanthin oxidase system whose effect is to generate superoxide radicals. Nitroblue tetrazolium, which is reduced into formasan blue in the presence of the free radicals produced by the hypoxanthin-xanthin oxidase system, is then used, and measurements of the optical density are performed, which enable an evaluation of the amount of radicals produced.

These operations are carried out in the absence and in the presence of the extract to be analyzed, and, by comparison of the results obtained, it is possible to evaluate the inhibition rate of the activity of the hypoxanthin-xanthin oxidase system in the presence of the extract to be analyzed.

In addition, in order to show that the observed anti-radical activity is not caused by the presence of alcohol in the cider, a 5% ethanol solution in water was tested in parallel, at the same 2.5% concentration (i.e., a final ethanol concentration of 0.125%). The obtained results are shown in the Table below.

| | Product | Ethanol | % inhibition | |
|---|---|---|---|---|
| | concentration | concentration | (% of control) | |
| Product tested | (%, v/v) | (%, v/v) | J0 | J30 |
| None (water | — | — | 0 | 0 |
| control) | | | | |
| Unfiltered extract | 2.5 | about 0.125 | 91 | 91 |
| Filtered extract | 2.5 | about 0.125 | 92 | 88 |
| 0.22 μm | | | | |
| 5% ethanolic | 2.5 | 0.125 | <10 | <10 |
| solution in water | | | | |
| (alcohol control) | | | | |

An inhibition in the order of 90% can be observed for an extract concentration of 2.5% in volume. In addition, it can be observed that this anti-radical activity is not linked to the presence of alcohol in the extract, although there is a light inhibition at less than 10%.

It can also be observed that this anti-radical activity is substantially unchanged when the extract is filtered beforehand by ultrafiltration on a sterilizing filter having a porosity of 0.22 μm. From this, it can be concluded that this activity is linked to totally soluble components.

Finally, it can be observed that this anti-radical activity is stable during at least one month.

Due to this anti-radical activity, the cosmetic products containing such an extract of at least one of the products obtained during valorization of the apple, in particular cider, have cosmetic anti-aging properties to protect the skin (anti-wrinkles, anti-oedema, anti-inflammation) or the phaneres (protection of the hair or of the nails).

Indeed, the harmful effect of the free radicals produced by solar radiations or pollution such as cigarettes, etc., is known. These effects act on the formation of wrinkles (alteration of dermis cells producing elastic and collagenic fibers, and degradation of the quantity and quality of these fibers), on oedemas (effect of the free radicals on the permeabilization of blood vessels, on the re-release of vasodilating molecules), on inflammation (induction of the production of inflammatory molecules that cause redness, oedema, itching and pain), on hydration (alteration of epidermis cells producing the epidermal lipids that give the epidermis its barrier function) and on hair and nails (alteration of the quality of keratin and hair scales).

1. Cosmetic product of the type cream, serum, gel, shampoo, bath, micro-emulsion or lotion to protect the skin or the phaneres, containing an extract of at least one of the products obtained during valorization of the apple in the context of making cider, such as the must constituted by juice extracted from the apple, one of the products of fermentation of the must.

2. Product according to claim 1, wherein said product which is the must strictly speaking after alcoholic fermentation.

3. Product according to claim 1, wherein said product is the must strictly speaking after malolactic fermentation.

4. Product according to claim 1, wherein said product is the sediment obtained after alcoholic fermentation of the must.

5. Product according to claim 1, wherein said product is the brown cap obtained after alcoholic fermentation of the must.

6. (canceled)

7. Method of manufacturing a cosmetic product for the protection of the skin or the phaneres, comprising using an extract of at least one of the products obtained during valorization of the apple, wherein said product or one of said products obtained during valorization of the apple is one of the following products: the must just after pressing of the apples, the must after alcoholic fermentation, the must after malolactic fermentation, the sediment, the brown cap, or the cider.
